(19) United States
(12) Patent Application Publication
(10) Pub. No.: US 2007/0023126 A1
(43) Pub. Date: Feb. 1, 2007

(54) Glass cart with RFID

(75) Inventor: Andy Sollers, Mogodore, OH (US)

Correspondence Address: TAROLLI, SUNDHELM, COVELL & TUMMINO, LLP, 1300 EAST NINTH STREET, SUITE 1700, CLEVELAND, OH, 44114, US

(73) Assignee: GED Integrated Solutions, Inc.

(21) Appl. No.: 11/493,118

(22) Filed: Jul. 26, 2006

Related U.S. Application Data
(60) Provisional application No. 60/703,167, filed on Jul. 28, 2005.

(51) Int. Cl. B32B 17/00; C12Q 1/68; B62B 1/00

(52) U.S. Cl. 156/104; 280/47.26; 435/6

(57) ABSTRACT

A glass cart includes a radio frequency identification tag that transmits a signal that can be received by a receiving unit to provide the location of the glass cart for tracking between manufacturing stations or facilities. Based on its position, a status of an order or a portion of a order carried by the cart is determined.

### CROSS REFERENCE TO RELATED APPLICATION

This application claims priority from U.S. Provisional Patent Application Ser. No. 60/703,167 file Jul. 28, 2005, entitled “GLASS CART WITH RFID” which is incorporated herein by reference.

### FIELD OF THE INVENTION

The present invention concerns a method and apparatus for tracking the location of carts carrying insulating glass units.

### BACKGROUND

Window manufacturers typically receive orders that include a variety of different sizes and types of windows and/or patio doors. The different sizes and types of windows and/or patio doors require different sizes and types of insulating glass units (IGs) that are assembled into a frame or sash to form a completed window or patio door at one or more glazing lines. Insulating glass units employed in windows and doors commonly are manufactured by sandwiching a peripheral spacer between aligned, parallel sheets of glass. The IGs are packaged and shipped to another location, often a separate manufacturing facility, in which the IGs are provided with appropriate frames to form finished windows and doors.

Glass carts have been developed for transporting sheets of glass and insulating glass units. These carts are designed to adequately protect the sheets of glass and IGs as they are transported between stations within a manufacturing facility or shipped via truck to a remote manufacturing facility. Due to the relatively high cost of the customized glass carts, the number of carts kept in service should be kept to a minimum.

### SUMMARY

A glass cart includes a radio frequency identification tag that transmits a signal that can be received by one or more receiving units to provide the location of the glass cart for tracking between manufacturing stations or facilities.

One exemplary method tracks a glass cart used in transporting insulating glass units or glass plates by storing a unique identifier in memory. In this embodiment the memory is physically mounted on a glass cart. A radio signal is transmitted that includes a unique identifier using a transmitter that is physically mounted on the glass cart. A receiver receives the radio signal and determines a position of the glass cart to be within a predetermined range of the position of the receiver. Based on its position, a status of an order or a portion of a order carried by the cart is determined.

These and other features of the invention will become further understood from a detailed description of an exemplary embodiment which is described in conjunction with the accompanying drawings.

### BRIEF DESCRIPTION OF THE DRAWINGS

FIG. 1 is a schematic depiction of an insulating glass unit manufacturing facility; and

FIG. 2 is a perspective view of a glass cart constructed in accordance with one embodiment of the present invention;

FIG. 2A is an enlarged view of an RFID tag that can be used in practice of one embodiment of the present invention.

### DESCRIPTION

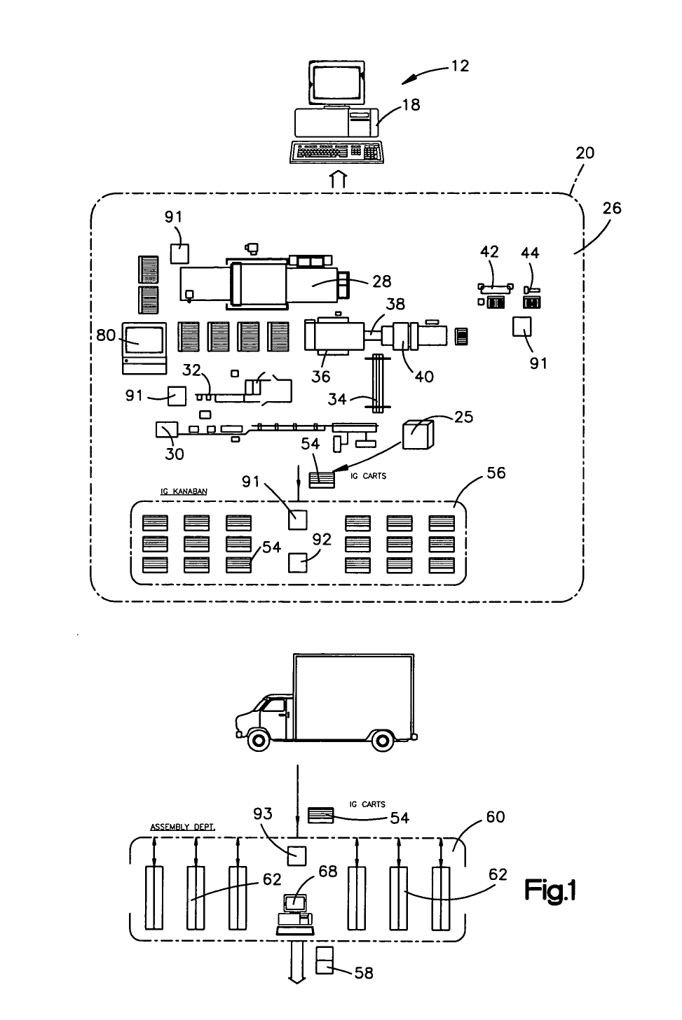

FIG. 1 schematically illustrates a window and/or door manufacturing facility 12. The facility 12 includes an insulating glass unit (IG) production control terminal 18. The production control terminal 18 receives orders for IGs and creates a sequence of runs of insulating glass units to be produced in an insulating glass unit (IG) department 26 that is part of an IG manufacturing facility 20. In the illustrated embodiment, the IG department includes several insulating glass component processing machines or stations that construct IG components and assemble the IG components to create IGs. Glass carts 54 are used to transport the IG components between stations. In the illustrated embodiment, the IG component processing stations include a glass cutting station 28, a spacer frame production station 30, a muntin bar production station 32, a muntin bar assembly station 34, a glass washing station 36, an IG assembly station 38, an oven 40, a gas fill station 42, and a patching station 44. Glass lites, spacer frames, and muntin bars are constructed at the glass cutting station 28, the spacer frame production station 30, and the muntin bar production station respectively. The glass lites, spacer frames, and muntin bar grids are assembled to form IGs at the IG assembly station 38. The IGs are fed through the oven/press 40, which presses the IG to a predetermined thickness and heats the adhesive/sealant that secures the lites to the spacer frame. The IGs are then filled with an inert gas at the gas fill station 42 and patched at the patching station 44.

Once patched, the finished IGs are placed on carts 54 and held in a staging area 56. The IGs are taken from the carts 54 and are assembled with window sash and frames or doors in a glazing facility 60. In the described embodiment the glazing facility 60 is a separate facility requiring transportation by truck of the glass carts 54. In the glazing facility completed windows and/or doors are constructed using the IGs on the glass carts. Assembly of an IG to a window or door sash and/or a frame is broadly referred to herein as glazing. The glazing facility 60 includes several discrete glazing lines 62.

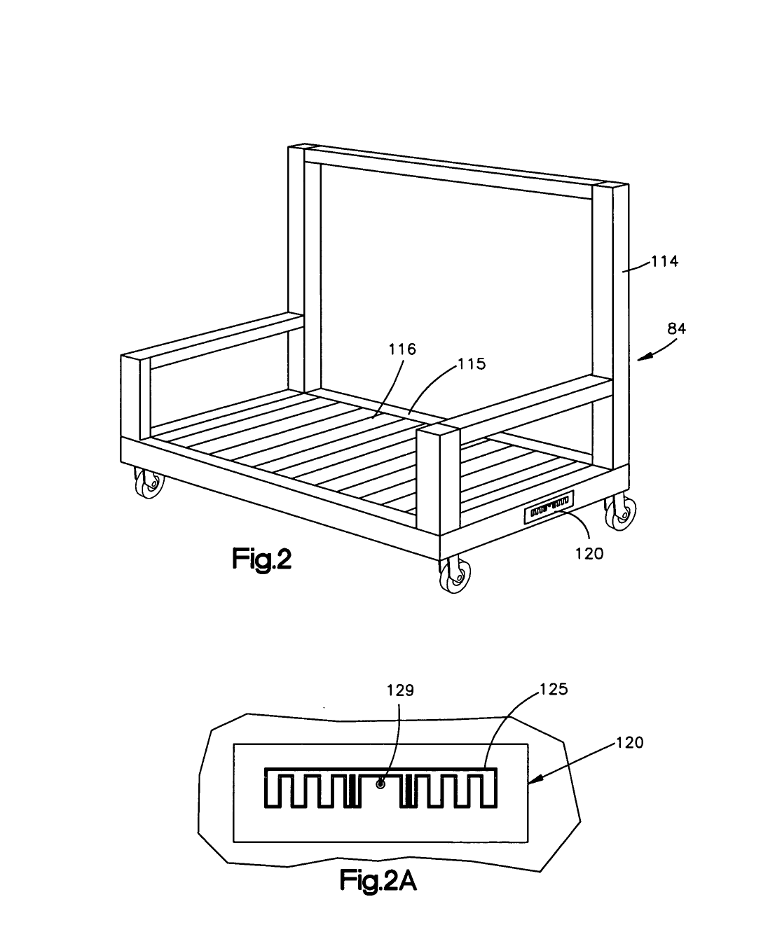

FIG. 2 illustrates a typical glass cart 54 that includes a frame 114 with a base 115 that has a number of parallel slots 116 configured to accept an IG or plate of glass. Other glass support mechanisms may be present but are not shown here. A radio frequency identification tag (“RFID tag”) 120 is attached to the glass cart. The RFID tag is used to track the location of the cart within either the IG manufacturing facility 20 or the glazing facility 60.

FIG. 2A is an enlarged view of one suitable RFID tag. The tag 120 includes a microchip 129 that stores data and an antenna 125. The tag 120 transmits a radio signal on a preselected frequency that is received by a reader (91 in FIG. 1). The radio signal includes a unique identification code that is associated with the particular cart on which the RFID tag is mounted. The tag 120 can be active or passive. Active tags are self powered and use a battery for power to send the signal. Passive tags receive power from the reader via a low power radio signal from the reader that is used to power the microchip. The tag is active relatively briefly, for the time required to verify the reader and exchange data. While active tags are more expensive than passive tags, they typically have a wider broadcast range.

Referring again to FIG. 1, several readers 91, 92, 93 are employed throughout the facility to track glass carts 54 within the IG manufacturing facility 20. Each reader sends and receives RF data to and from the RFID tags on the glass carts 54. Each reader may have multiple antennas that are responsible for sending and receiving the radio waves. Typically, RFID readers and tags operate on one of three primary frequency bands: low frequency (125/134 KHz), mid-frequency (13.56 MHz), and ultra high-frequency (850 MHZ-950 MHz and 2.4 GHz-2.5 GHz).

It may be advantageous to provide at least one reader at each manufacturing station so that the location of a given glass cart 54 and the glass panes or IGs that it carries can pinpointed and its progress through the stations logged. An order for finished IGs can be assigned a glass cart identification code corresponding to the cart on which the unfinished glass panes that will be used for the IGs are placed. As the glass cart is moved to a new station, the radio signal from the tag is read by the reader associated with that station. In this manner, the location of the glass cart can be tracked as well as the status of the order held by the glass cart.

As discussed above, finished IGs can be stored in a staging area 56 until they are needed by the glazing facility 60. A staging area reader 92 receives signals from carts as they pass out of the staging area and leave the IG manufacturing facility 20. When the glass cart enters the glazing facility 60, a glazing facility reader receives a signal from the RFID tag on the cart and logs the presence of the cart within the facility in a production control computer 68. In this manner, the number of glass carts that are present in either the IG manufacturing facility or glazing facility can be known at any given time.

While several embodiments of the invention has been illustrated and described in detail, the present invention is not to be considered limited to the precise constructions disclosed. Various modifications, adaptations and uses of the invention may occur to those skilled in the art to which the invention relates. The intention is to cover all such modifications, adaptations and uses falling within the spirit or scope of the claims.

1. A glass cart comprising:
a frame with a base configured to support one or more insulating glass units or plates of glass;
a circuit comprising memory for storing a unique identifier associated with the glass cart and instructions for transmitting radio signals corresponding to the unique identifier; and
an antenna for transmitting the signals corresponding to the unique identifier over a preselected frequency.

2. The glass cart of claim 1 comprising a power source for powering the microchip.

3. The glass cart of claim 2 wherein the power source is a battery.

4. The glass cart of claim 2 wherein the power source converts a radio signal received by the antenna into power for the microchip.

5. A method for tracking a glass cart comprising:
storing a unique identifier in a memory and mounting the memory on a glass cart;
transmitting a radio signal that corresponds to the unique identifier using a transmitter that is physically mounted on the glass cart and transmits in a selected frequency;
receiving the radio signal with a receiver and determining a position of the glass cart to be within a predetermined range of the position of the receiver.

6. The method of claim 5 comprising mapping a location to each of a plurality of receivers and determining the glass cart to be at the location mapped to the receiver that receives the radio signal from the glass cart.

7. The method of claim 5 comprising transmitting a power signal to the transmitter to power the transmitter.

8. A glass cart tracking system comprising:
a computer memory mounted on each glass cart that stores a unique identifier associated with the glass cart;
a transmitter mounted on each glass cart that transmits signals indicative of the unique identifier;
a receiver that is mapped to a location that receives the transmitted signal and indicates that the glass cart is within a given range of the mapped location.

9. The glass cart tracking system of claim 8 comprising a power source that powers the transmitter.

10. The glass cart tracking system of claim 8 wherein the power source is a battery.

11. The glass cart tracking system of claim 8 wherein the power source is a converter that converts a radio signal into power for the transmitter.
